(19) United States
(12) Patent Application Publication
(10) Pub. No.: US 2021/0002598 A1
(43) Pub. Date: Jan. 7, 2021

(54) METHODS OF MIXING AND DISPENSING CELLS

(71) Applicant: PBS Biotech, Inc., Camarillo, CA (US)

(72) Inventors: Chanyong Brian Lee, Newbury Park, CA (US); Yasunori Hashimura, Woodland Hills, CA (US); Oscar Garza, Camarillo, CA (US); Gary Evans, Camarillo, CA (US)

(21) Appl. No.: 16/460,132

(22) Filed: Jul. 2, 2019

(51) Int. Cl. C12M 3/06; C12N 5/071; C12M 1/06; C12M 1/36

(52) U.S. Cl. CPC C12M 27/14; C12M 41/48; C12M 27/02; C12N 5/0676

(57) ABSTRACT

A vessel having a mixer that ensures a homogeneous cell distribution in dispensed quantities. The vessel has a mixer therein for stirring contents of the vessel and an orifice in a lower wall to which a cell dispenser is attached. The cell dispenser dispenses quantities of suspended cells having a homogeneous cell distribution. The vessel may be a closed system with no option or instructions for removing a cap or other access port, whereby after manufacturing the vessel is shipped to a customer and the interior remains closed to the exterior environment and fluids are transferred through tubes.

### TECHNICAL FIELD

The invention pertains to vessels for dispensing cultured cells suspended in fluid and, more particularly, to a closed system vessel having a mixer for dispensing quantities of cells suspended in fluid having a homogeneous cell distribution, and to methods of dispensing quantities of cells.

### BACKGROUND OF THE INVENTION

In the conventional therapeutic protein-based industry, recombinant cells are expanded and induced to produce target proteins, which are then isolated and purified before final formulation in chilled excipient designed to stabilize proteins. In such application, maintaining proteins in uniform suspension in the final fill/finish step is not a great concern, largely due to the fact that proteins do not settle very fast in the excipient relative to the time required for processing to create noticeable concentration gradient.

On the other hand, in the growing field of cell therapy where animal cells in their native pluripotent, induced pluripotent, and/or differentiated form would be cultured and expanded, the cells themselves are the final product that must be isolated and dispensed into final vials. Maintaining cells in uniform suspension in the excipient during the dispensing step is more critical and challenging compared to maintaining proteins in suspension. Although there are a number of ways to dispense such cells in the art, such as withdrawing cells with a pipette from above, none as yet has been able to repeatedly and accurately dispense cultured cells from a vessel on demand.

### SUMMARY OF THE INVENTION

The present application discloses a preferably closed system vessel having a mixer that ensures a homogeneous cell distribution in dispensed quantities.

An appreciation of the other aims and objectives of the present invention and an understanding of it may be achieved by referring to the accompanying drawings and the detailed description of a preferred embodiment.

### BRIEF DESCRIPTION OF THE DRAWINGS

FIG. 1 is a perspective view of an embodiment of the homogeneous cell-dispensing mixer;

FIG. 2 is a close-up sectional view of a dispensing portion of the mixer;

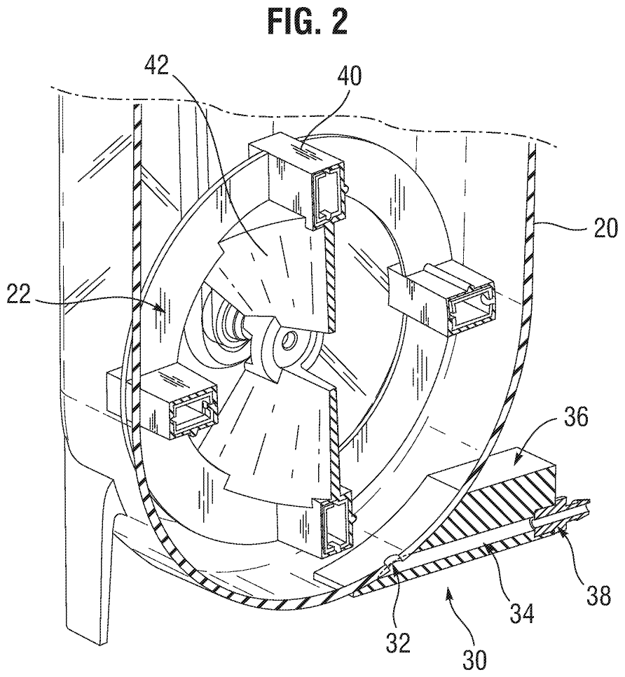

FIG. 3A is a perspective view of an embodiment of a closed system homogeneous cell-dispensing mixer; and

FIGS. 3B and 3C are front and side elevational view of the closed system mixer of FIG. 3A.

### DESCRIPTION OF THE PREFERRED EMBODIMENTS

The present application relates to vessels for dispensing cells suspended in fluid and, more particularly, to a vessel having a mixer that ensures a homogeneous cell distribution in dispensed quantities.

In the growing field of cell therapy the final products are animal cells in their native pluripotent, induced pluripotent, and/or differentiated form. The cells themselves must be isolated and dispensed into final vials. Maintaining cells in uniform suspension in the excipient during the dispensing step is much more critical and challenging compared to maintaining proteins in suspension. This is due to the faster settling velocity of cells, the relatively large size of the cells (micrometer scale vs. nanometer scale) which limits the minimum size of the orifice required for accurate and low-shear dispensing, and the higher shear sensitivity level of cells which can impact the viability of cells dispensed.

Further, the cell-dispensing step requires that a sterile vessel be used to mix the animal cells and excipient at a controlled refrigeration (2-8° C.) temperature and in an aseptic manner to ensure that the cell product is not contaminated with foreign particles or microorganisms. Typical lot release criteria for this cell-dispensing step are that the vials selected for QC inspection must meet a target cell concentration that fall within acceptable tolerance and that they must meet a minimum viability target. The process requires that cells be dispensed in accurate volume, at accurate cell concentration, within short processing time, and at controlled 2-8° C. temperature to ensure uniformity in cell concentration and high cell viability in the vials.

This proposed solution for dispensing such animal cells includes a vessel for containing the cell suspension having a mixing device that allows the cells to be maintained in uniform suspension during dispensing at 2-8° C. condition into vials in a relatively low-shear manner to avoid damaging cells. The device would consist of a mixing vessel to hold the cells and excipient in a sterile manner, with an impeller that is rotated by any number of means—pneumatically, magnetically, or otherwise—to keep the cells suspended uniformly in the excipient. The rotational speed of the impeller should be controllable by the user in a repeatable manner and to the extent that would allow the cells to be suspended uniformly and dispensed within allowable tolerance.

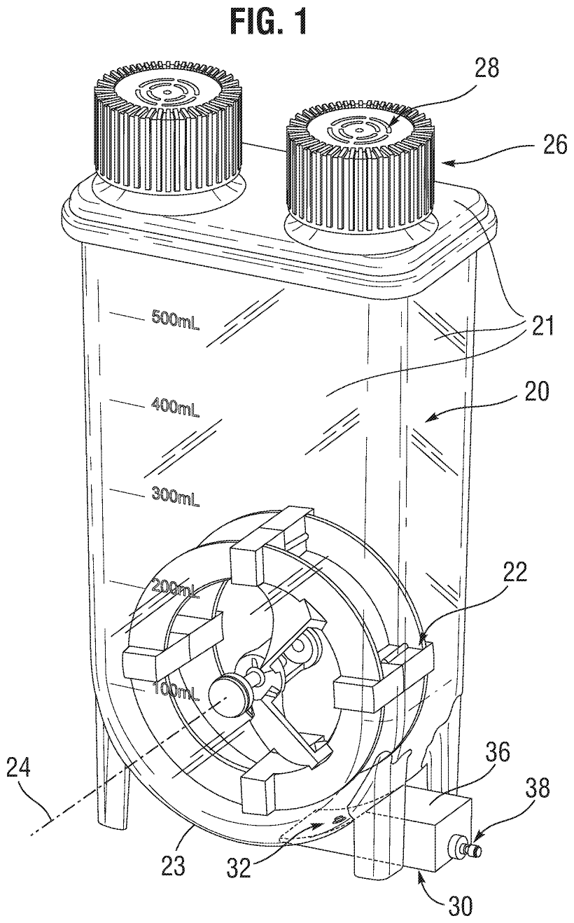

One embodiment of this invention, as depicted in FIG. 1, comprises a vessel 20 defined by outer walls 21 to hold the chilled cells and excipient and an impeller 22 enclosed within the vessel for maintaining cells in suspension. The outer walls 21 include a lower curved wall 23. The impeller 22 is positioned in a lower portion of the sterile containment vessel and oriented in a vertical plane and rotates about a horizontal axis 24 to allow maximum particle suspension at minimum power input and reduce shear effects on cells. Cells and excipient are introduced into the vessel by removing a threaded port cap 26 in a Class 100 clean room environment or equivalent, and then transferring the content into the vessel 20 via pipetting or pouring. The cap 26 may be threaded back onto the port to seal prior to cell dispensing to minimize potential for introducing foreign materials. A hydrophobic membrane 28 on the cap 26 allows improved thermal exchange with the air in the cold room to help maintain temperature.

During cell dispensing, fluid is removed at a lower dispenser 30 via a vessel aperture or orifice 32 that extends through an outer wall near the bottom of the vessel 20. The fluid travels down a bore 34 in a machined block 36 of the dispenser 30 which is affixed to the vessel 20 and sealed around the orifice 32. A hose barb adaptor 38 open to the bore 34 that mates with the machined block 36 allows tubing to be secured to it to maintain a sterile fluid path. Prior to sterilization of this device, tubing would be attached and secured to the hose barb adaptor 38 and terminated with another adaptor depending on how the user wishes to connect it to a dosing pump (not shown).

The impeller 22 consists of a plurality of paddles 40 along its outer periphery that generate strong sweeping motion of the liquid as it rotates to counteract cell settling in the excipient. The paddles 40, which are hollow, can encapsulate permanent magnets, which are used to couple with magnets on the agitation controller (not shown) to drive the rotation of the impeller 22. The impeller 22 also consists of two diametrically-opposed vanes 42 extending from the paddles to an inner hub that create bi-axial fluid flow as the impeller rotates to ensure homogeneity of cells suspended in the excipient. That is, the vanes 42 have curved surfaces that urge flow axially when the impeller is rotated in one direction.

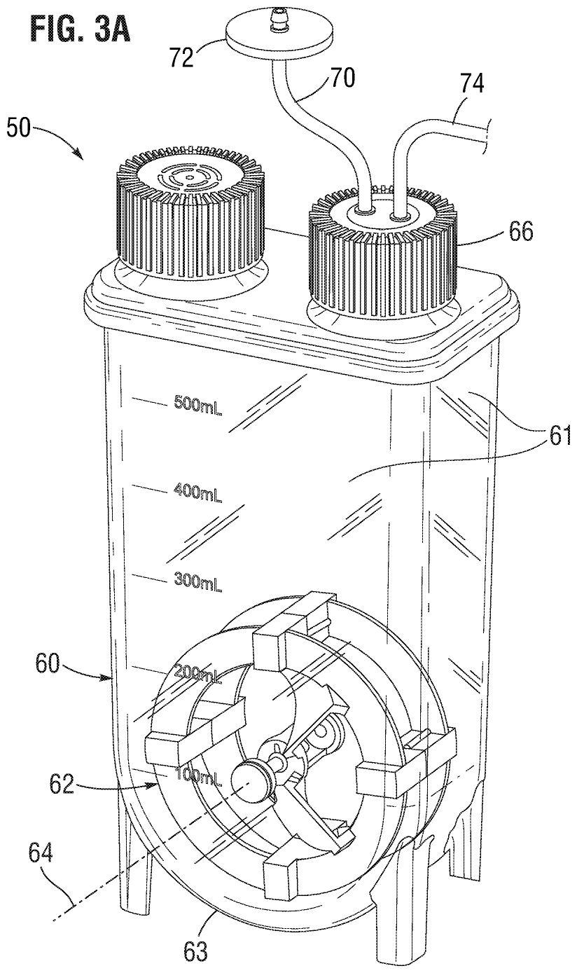

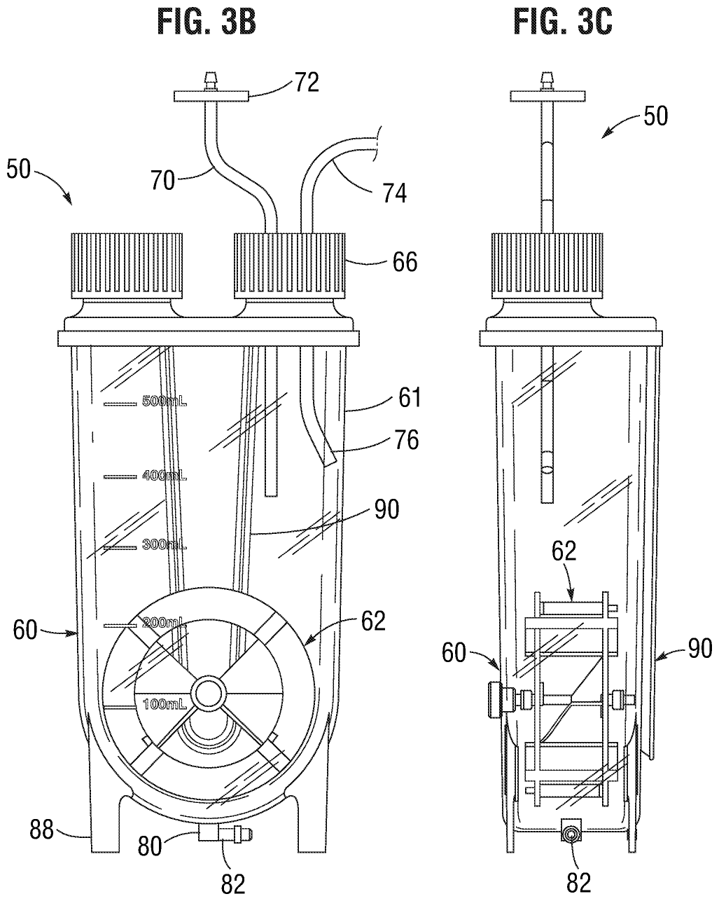

FIG. 3A is a perspective view of another embodiment of the homogeneous cell-dispensing mixer 50, and FIGS. 3B and 3C are front and side elevational view thereof. The mixer 50 again comprises a vessel 60 defined by outer walls 61 to hold the chilled cells and excipient and an impeller 62 enclosed within the vessel for maintaining cells in suspension. The outer walls 61 include a lower curved wall 63. The impeller 62 is positioned in a lower portion of the sterile containment vessel and oriented in a vertical plane and rotates about a horizontal axis 64 so as to sweep closely past the lower curved wall 63 and produce maximum particle suspension at minimum power input and reduce shear effects on cells.

The mixer 50 in this case is a closed system which means there is no option or instructions for removing a cap or other access port. Closed in this sense means that from the moment manufacturing is complete and the vessel 50 is shipped to a customer, the interior remains closed to the exterior environment. As will be seen, there are still ways to input and output fluids, cells, and other media for operating the bioreactor, but those means are constrained to tubes and the like which remain closed off until connected with another closed source or dispensing chamber.

Cells and excipient are introduced into the vessel through ports in a threaded port cap 66 in a Class 100 clean room environment or equivalent. Rather than removing the cap 66, cells and excipient may be added or removed through access tubes that pass through ports in the cap. Specifically, an add tube 70 passes through a sealed port in the cap 66. The add tube 70 has an air filter 72 incorporated therein for venting displaced air from within the vessel 60 as fluid is added. A feeding tube 74 passes through a sealed port in the cap 66 for adding liquid media containing cells to the vessel 60. Since the mixer 50 is a closed system, the threaded port cap 66 is delivered to the customer in a non-removable state, such as being adhered or heat bonded to the upper lid.

As seen in FIG. 3B, the feeding tube 74 has a lower end 76 curved towards the interior of a vessel wall 61, which will prevent liquid media containing cells from dropping straight down onto the impeller wheel 62. Adding media containing cells directly onto the impeller wheel 62 might cause cell damage, or splashing into existing media as the vessel fills up and/or scattering cells high up on the walls 61. The angle of the feeding tube 74 at the lower end 76 is desirably between about 10-20°. The feeding tube 74 may connect or be sterile welded to a source tube.

In contrast with the first embodiment, a dispenser in the form of a bottom port 80 replaces the machined block 36. The bottom port 80 may comprise a fitting welded or adhered to an aperture or orifice at the lower nadir of the lower curved wall 63. Desirably, no part of the bottom port 80 projects upward into the vessel 60 interior to avoid creating a flow disturbance. A connector nipple 82 may be provided that angles 90° from the bottom port 80 for connection of supplemental tubing. The connector nipple 82 may terminate in a hose barb adaptor as with the adaptor 38 described above. Legs 88 extend a short distance down from the lower curved wall 63 of the vessel 60 to provide a small space for connection of the supplemental tubing. In addition, a V-shaped molded bracket 90 may be formed in a rear wall of the vessel 60 that fits closely within a similarly-shaped cavity in a larger housing that receives and contains the vessel. Such a housing preferably has a large front window for viewing the reaction process and connections for the various fluid inputs and outputs and electronic monitoring and control equipment.

Desirably, there is a minimum of one port for adding cells and excipient into the vessel and a minimum of one port for dispensing the cells and excipient, both of which could be sealed as needed to prevent foreign contaminants, biological or not, from contacting the cell product. The dispensing port should allow for flexibility by the user to specify how to connect the device to a dosing pump—either by using an aseptic connector (GE ReadyMate Disposable Aseptic Connector, Pall Kleenpak™ Sterile Connector, or equivalent), a dead-ended thermoplastic tubing that may be heat welded onto another dead-ended thermoplastic tubing, or tubing that is terminated with fittings that may be connected to another tubing inside a Class 100 clean room environment. The dosing pump would be a calibrated instrument to allow accurate metering of liquid dispensed into vials.

Since a temperature of 2-8° C. would be maintained in the vessel either by placing the mixing device in a cold room or a refrigerator or by applying cold packs, the vessel wall would therefore be composed of a material and thickness that allows relatively high thermal transfer. If the addition port is positioned at the top of the vessel, the cap on the port could further contain a hydrophobic, sterilizing-grade (0.22-micron or finer) membrane to allow gas exchange with chilled gas in the cold room or refrigerator for improved thermal transfer. Additionally, the material could be clear in appearance to allow visual confirmation of impeller rotation and cell suspension.

All of the components of this mixing device that come in contact with the chilled excipient and cells should be manufactured from medical-grade materials that have been certified to USP Class VI, ISO 10993, or equivalent, to ensure they meet the regulatory requirements of the user. The mixing device would also need to be sterilizable to ensure Sterility Assurance Level (SAL) of 10⁻⁶—either by gamma radiation, steam sterilization, or other applicable means.

It is understood that the foregoing examples are considered illustrative only of the principles of the invention. Further, since numerous modifications and changes will readily occur to those skilled in the art, it is not desired to limit the invention to the exact construction and operation shown and, accordingly, all suitable modifications and equivalents may be resorted to, falling within the scope of the invention.

1-20. (canceled)

21. A media dispensing device, comprising:
a closed sterile containment vessel having:
i. outer walls comprising a lower curved wall located at a lower end of the sterile containment vessel,
ii. a vent filter,
iii. a mixer in the sterile containment vessel configured to rotate about a horizontal axis and positioned in a lower portion of the sterile containment vessel so as to stir contents of the sterile containment vessel adjacent the lower curved wall, and
iv. an orifice in the lower curved wall; and
an outlet port in communication with the orifice and configured to connect to tubing.

22. The media dispensing device of claim 21, wherein the sterile containment vessel is closed to an external environment.

23. The media dispensing device of claim 21, wherein the vent filter allows bi-directional flow of air.

24. The media dispensing device of claim 21, wherein the sterile containment vessel comprises a lid.

25. The media dispensing device of claim 24, wherein the lid and the vent filter are spaced from one another.

26. The media dispensing device of claim 21, wherein the vent filter comprises an air filter.

27. The media dispensing device of claim 21, wherein the sterile containment vessel comprises an inlet port that is closed off.

28. The media dispensing device of claim 21, wherein the outlet port is closed off.

29. The media dispensing device of claim 21, wherein the sterile containment vessel comprises an inlet port having a non-removable port cap affixed to the sterile containment vessel.

30. The media dispensing device of claim 21, further comprising an add tube in communication with the sterile containment vessel, the vent filter being coupled to the add tube.

31. The media dispensing device of claim 21, further comprising a feeding tube in communication with the sterile containment vessel for adding media to the sterile containment vessel.

32. The media dispensing device of claim 31, wherein the feeding tube comprises a first section extending out of the sterile containment vessel and a second section extending into the sterile containment vessel.

33. The media dispensing device claim 31, wherein the feeding tube comprises an angled lower end disposed inside of the sterile containment vessel.

34. The media dispensing device of claim 33, wherein the angled lower end terminates in an opening that is disposed in close proximity to a sidewall of the sterile containment vessel and configured to substantially limit media from at least one of dropping straight down onto the mixer, splashing, or foaming.

35. The media dispensing device of claim 33, wherein the angled lower end of the feeding tube has an angle relative to an adjacent portion of the feeding tube.

36. The media dispensing device of claim 21, wherein the media comprises a liquid media.

37. The media dispensing device of claim 21, wherein the media comprises cells.

38. A media dispensing device, comprising:
a closed sterile containment vessel;
a mixer disposed in the sterile containment vessel and configured to rotate about a horizontal axis;
a vent filter adapted to vent displaced air from within the sterile containment vessel;
a feeding tube extending into the sterile containment vessel and having an angled lower end that curves toward an interior surface of a sidewall of the sterile containment vessel to substantially prevent media from at least one of dropping straight down onto the mixer, splashing, or foaming; and
an outlet port coupled to the sterile containment vessel and configured to connect to tubing for dispensing mixed media from the sterile containment vessel.

39. The media dispensing device of claim 38, wherein the sterile containment vessel is closed to an external environment.

40. The media dispensing device of claim 38, wherein the sterile containment vessel comprises an inlet port that is sealed for sterility.

41. The media dispensing device of claim 38, wherein the outlet port is sealed for sterility.

42. The media dispensing device of claim 38, further comprising a non-removable port cap affixed to the sterile containment vessel and coupled to at least one of the vent filter or the feeding tube.

43. The media dispensing device of claim 38, further comprising tubing coupled to the outlet port.

44. The media dispensing device of claim 38, wherein the media comprises cells.
